(19) United States
(12) Patent Application Publication
(10) Pub. No.: US 2008/0071299 A1
(43) Pub. Date: Mar. 20, 2008

(54) Bioabsorbable elongated member

(75) Inventors: Timo Allinniemi, Lempaala (FI); Pertti Tormala, Tampere (FI); Esa Partio, Sipoo (FI)

Correspondence Address: VENABLE LLP, P.O. BOX 34385, WASHINGTON, DC, 20043-9998, US

(73) Assignee: BIORETEC OY, Tampere (FI)

(21) Appl. No.: 11/902,308

(22) Filed: Sep. 20, 2007

(30) Foreign Application Priority Data
Sep. 20, 2006 (FI) 20065574

(51) Int. Cl. A61B 17/08; A61B 17/00

(52) U.S. Cl. 606/151; 606/86

(57) ABSTRACT

A bioabsorbable elongated member including a first end and a second end and an elongated part therebetween. The elongated part includes a front end, a back end and a target point in which the front end terminates and the back end begins. The bioabsorbable elongated member possesses a predetermined orientation. The orientation of the bioabsorbable elongated member is alterable so that a bulge is formed in the bioabsorbable elongated member by mechanical, thermal or thermomechanical straining of the elongated member at the target point. The bulge forms a local stopper of the bioabsorbable elongated member, or a part of a local stopper, or a bending point of the bioabsorbable elongated member. A method for forming a local stopper, a part of a local stopper, or a bending point in a bioabsorbable elongated member.

### FIELD OF INVENTION

The present invention relates to a bioabsorbable elongated member which may be used in a bone and/or tissue fixation.

### BACKGROUND OF THE INVENTION

In bone surgery it is well known to use metallic tension band wires in the internal fixation of bone fractures, osteotomies and pseudarthroses. A typical tension band wire is a flexible metallic wire with a diameter of about 1 mm. The clinical use of tension band wires is described e.g. in M. E. Müller et al., “Manual of Internal Fixation”, Springen-Verlag, Berlin Heidelberg New York, 1979, pages 42-47. However, tension band wiring has several drawbacks. Because of the high tensile modulus of metallic wires, the tightening of wire too much can lead to bone fractures or to necrosis under the wire. Also the knotting of the wire loop creates a bulky knot which can irritate tissues, especially subcutaneous tissue on a bone, which can lead even to an infection and/or sinus formation.

To eliminate wire knotting and its problems, several types of connectors or locking implants (locking systems) have been developed to hold surgical wires, bands, cables etc. in desired position around and/or inside of bone. Common forms of locking members and systems are e.g. crimps (e.g. U.S. Pat. No. 5,536,270, U.S. Pat. No. 5,649,927), connectors (U.S. Pat. No. 5,415,658), cable ties (U.S. Pat. No. Des. 369,960, U.S. Pat. No, 3,886,630), loop locking structures (U.S. Pat. No. 4,813,416), suture locks (U.S. Pat. No. 5,364,407), clamps (U.S. Pat. No. 4,201,215), buckles (U.S. Pat. No. 5,355,913) and locking systems comprising bioabsorbable fasteners (U.S. patent application Ser. No. 10/657,087).

However, current designs of (especially metallic) locking members and systems, particularly for surgical use, have significant drawbacks. For example, a tension which is too high may lead to improper healing or poor medical results (such as a bone fracture and/or necrosis).

Also, in many prior art locking systems there is the risk of the slippage of the wire or cable in relation to the locking member, which can lead to delayed healing. The loosening of the cable, e.g. in bone fracture fixation, can also lead to delayed healing, pain or even to a failure to heal. Also, many prior art locking members cannot be locked easily at a desired tension, and often the retained tension cannot be maintained when using a cable loop or winding as there is an inevitable drop in the tension when the pliers are removed. Thus, a surgeon typically has to “overshoot” the desired tension, approximating how much of that tension will be lost after the locking has been completed and the pliers have been removed thus significantly increasing the risk of tensioning errors.

In addition, many prior art locking members are bulky and may cause adverse tensioning in the surrounding tissues, which may result in a negative effect on tissue healing. Another drawback of many prior art cable and locking member systems is that they are made of metal, such as stainless steel. Such extremely stiff materials are mechanically incompatible with bone tissue and therefore, they may cause osteolysis around the material, which may lead to implant migration.

Consequently, it is desirable to use less stiff band and locking member systems, thereby preventing osteolysis and implant migration. It may also be desirable to use bands and locking member systems in which band slippage in relation to the locking member(s) is not possible. It may also be desirable to use bioabsorbable band and locking member systems so that the implant will absorb after healing of the bone fracture or osteotomy.

U.S. patent application Ser. No. 10/657,087 describes locking systems comprising bioabsorbable fasteners. However, drill holes must be drilled into the bone to fix the fasteners and the bioabsorbable band together. The drilling of additional drill holes into the bone creates an additional trauma to the patient and prolongs the duration of the surgical operation.

Therefore, there is still a need for a minimally invasive fixation device which is simple, easy to make and rapid and easy to use surgically. There is also a need for a fixation device in which the locking system is small but creates a secure locking and fixation of bone fragment(s).

### SUMMARY OF THE INVENTION

The present invention provides a bioabsorbable elongated member which provides accurate tensioning of the band and safe locking of the band around and/or inside of the bone by means of minimally invasive surgery.

The present invention also provides a bioabsorbable elongated member which is simple, easy to make and rapid and easy to use surgically.

The present invention also provides a bioabsorbable elongated member which is small but creates a secure locking of the member and fixed bone fragments.

The orientation of the elongated member of the invention is locally alterable, usually the elongated member is radially expandable. The radial expansion is a consequence of the microstructure of the implant of the invention. At least a part of the elongated member is constructed of a material having uniaxial, longitudinal stresses, formed therein by solid state drawing or pullforming. Multiaxial stresses are also possible. The multiaxial stresses are created for example by turning spirally a blank of the elongated member. These stresses in the material, when treated mechanically (such as by locally moving around and bending), thermally or thermomechanically, are relieved, whereby a radial, local expansion of the material takes place, thus creating a bulge.

The property of creating the bulge can be identified simply by bending the bioabsorbable elongated member by hand repeatedly at the same point. The material of the bioabsorbable elongated member heats up due to the internal friction while it is bent. After a few bends, the bulge forms due to the relaxation of the material. Shortly after the bulge has formed, the bioabsorbable elongated member can be bent at the bulge in a desired direction, or the bulge can be left as such in order to form a local stopper, or a part of a local stopper. It is naturally also possible to move the elongated member in other ways than by bending; all mechanical treatments, for example rotating, having the same result, i.e. the bulge, are usable as well.

The main benefit of the mechanical treatment compared to the other possible methods is that the bulge can be formed in-situ during a surgical operation without special tools, i.e. one can freely decide where to form the bulge, and it can be done by hand simply for example by bending the elongated member back and forth at the same point. The bulge may be used as a stopper, a part of a stopper, or a bending point of a bioabsorbable member. The bulge may be used instead of knots, or other stoppers or locking members.

The other possible methods for forming a bulge include thermal or thermomechanical treatments. However, such treatments require a tool for forming the bulge. The tool may be a heating device, or a device which combines heating and mechanical treatment, such as heatable pliers.

It is possible to use a tightening ring, a retaining plate, or a cannulated screw to secure that the bulge remains in its position if there is a risk that the elongated member may move.

In an embodiment of the present invention, a bioabsorbable member for securing a bone fracture, or bone fractures, is provided, comprising a first end, an elongated part and a second end. The elongated part may be flexible or rigid. The bioabsorbable member can be locally expanded radially by means of mechanical or thermal or thermomechanical treatment. The mechanical treatment is preferred.

In another embodiment of the present invention, a method to secure bone fracture(s) or osteotomy (osteotomies) with a bioabsorbable elongated member is provided. The method includes (a) the radial expansion of a part of a first end part to create a first locking member (local enlargement of the first end part), (b) pushing a second end part (equipped with an optional needle) and a shaft part into and through drill hole(s) in bone fragment(s), (c) tightening the elongated member by pulling the second end part which is outside of bone after pushing through drill hole(s) and (d) the local radial expansion of the second end part under tension just on the opening of the drill hole from which the second end part emerges, in order to create a second locking member (local enlargement of the second end part) and (e) cutting of the first end part and second end part so that the first locking member remains in the entrance opening of the first drill hole and the second locking member remains on the outlet of the last drill hole.

In another embodiment of the present invention, a method to secure a bone fracture or osteotomy with a bioabsorbable elongated member is provided. The method includes (a) passing the second end part (optionally equipped with a needle) around the bony tissues to be drawn together, (b) pulling the second end part through a hole in the (optionally flattened) part of the first end part (now the first locking element), (c) tightening the elongated member by keeping the first end part in place and pulling the second end part to a proper tension, (d) expanding locally and radially the second end part under tension to create the second locking element (local enlargement of the second end part) just outside of the outlet of the hole in the first locking element, and (e) cutting the second end part so that the second locking element secures the closing of the band loop surrounding the bony tissues fixed together.

The bioabsorbable implants (bioabsorbable bands or rods) of this invention may be manufactured of bioabsorbable polymers, copolymers or polymer mixtures or alloys. The preferred material is a copolymer of lactide and glycolide. The material may comprise 70 to 85 wt.-% lactide and 15 to 30 wt.-% glycolide. Suitable manufacturing methods include molding, sintering and/or solid state deformation (pullforming) methods which are described e.g. in U.S. Pat. No. 4,743,257, U.S. Pat. No. 4,968,317, EP Pat. No. 0423155, AU Pat. No. 729801, EP Pat. No. 1009448 and in U.S. Pat. No. 6,406,598.

The strong and tough oriented structures are especially advantageous in implant systems of this invention. They may be created also during extrusion or injection molding of absorbable polymeric melt through a suitable die or into a suitable mold at high speed and pressure. When cooling occurs at suitable conditions, the flow orientation of the melt may remain in the solid material as an oriented structure. In an advantageous embodiment, the mold may have the form of the final device, but it is also possible to manufacture the implants of the invention by machining (possibly using also heat) and by thermoforming of injection-molded or extruded semi-finished products.

It is advantageous to make the implants of melt-molded, solid state drawn or compressed, bioabsorbable polymeric materials, which are described e.g. in U.S. Pat. Nos. 4,968,317 or 4,898,186.

The reinforcing fibers of the implants may also be ceramic fibers, such as bioabsorbable hydroxyapatite or bioactive glass or tricalcium phosphate fibers. Such bioabsorbable, ceramic fiber reinforced materials are described e.g. in European Patent Application No. 0146398 and in WO 96/21628.

The oriented and/or fiber reinforced implants of this invention may be manufactured by molding the reinforcement fiber-polymer matrix to the final product in a mold whose mold cavity has the form of the final product, or the final form may be machined mechanically (possibly also using heat) of a preform, such as a melt-molded and solid-state drawn rod, as described e.g. in U.S. Pat. No. 4,968,317.

The reinforcement elements may extend into any protrusions or ridges of the implant. The reinforcement elements may also turn spirally around the longitudinal axis of the implants. Also other different orientations of reinforcement elements in elongated samples which are known in composite technology may be applied to the present invention. However, a general feature of the orientation and/or fiber-reinforcement of the implants of this invention is that many of the reinforcing elements are oriented in such a way that they can carry effectively the different external loads (such as tensile, bending and shear loads) that are directed to the healing bone fracture or osteotomy, e.g. sternotomy.

According to an advantageous embodiment of the invention, the implant, or a special coating layer on its surface, may contain one or more bioactive substances, such as antibiotics, chemotherapeutic substances, growth factors such as bone morphogenic proteins, substances accelerating the healing of the wound and osteotomy, hormones, antibiotics or other drugs and the like. Such bioactive implants are especially advantageous in surgical use, because they contribute biochemically to the healing of the lesion in addition to providing mechanical support.

The oriented and/or reinforced materials of the implants of this invention typically have initial tensile strengths of about 100 to 1000 MPa, bending strengths of about 100 to 500 MPa and shear strengths of about 80 to 300 MPa. The implants can be made stiff, tough, and/or flexible. These mechanical properties are superior to those of non-oriented and non-reinforced absorbable polymers which typically show strengths between 40 and 100 MPa and may additionally be brittle (see e.g. S. Vainionpää, P. Rokkanen and P. Törmälä, “Surgical Applications of Biodegradable Polymers in Human Tissues”, Progr. Polym. Sci 14/1989, pp. 679-716).

A special advantage of the present invention is that there are no bulky crimps in these implants. They can be made relatively thin e.g. with a cylindrical shaft part having a diameter between 0.5 and 4 mm and a bulge having a diameter between 1 and 8 mm.

The implants of the present invention may be sterilized by any of the well known sterilization techniques, depending on the type of material used in manufacture of the implant. Suitable sterilization techniques include radiation sterilization such as cobalt 60 irradiation or electron beams, ethylene oxide sterilization, and the like.

The elongated member may be provided with at least one needle. Instead of the needle there may also be a drill bit or a kirschner wire by which a drillhole can be drilled into a bone.

The possible uses of the elongated member comprise, for example, olecranon fractures, patella fractures, tuberculum majus fractures of proximal humerus, comminuted fractures of distal tibia (also syndesmosis) and comminuted proximal tibia fractures. In reconstructive plastic surgery the elongated member can be used for the fixation of bone containing a latissimus dorsi graft. The elongated member may be used in combination with other pins and screws.

### BRIEF DESCRIPTION OF THE DRAWINGS

In figures only FIGS. 1b, 1c, 9e-9i, 12b show a bone to be fixed. However, a person skilled in the art will readily understand how the bone or tissue exists with respect to an elongated member in other figures.

In the figures,

FIG. 1a shows an elongated member in a perspective view,

FIGS. 1b and 1c show the elongated member inserted in a bone (the bone is shown in a cross-sectional view in order to show the elongated member,

FIGS. 2a to 8c show variations of elongated members in perspective views,

FIGS. 9a to 9d show an elongated member in a perspective view,

FIGS. 9e to 9j show a schematical view how the elongated member of FIG. 9a is inserted in a cranium,

FIGS. 10a to 10d show an elongated member in a side view,

FIGS. 11a to 11d show possible head designs of an elongated member,

FIG. 12a shows fractures in the distal end of a tibia,

FIG. 12b shows the distal end of the tibia of FIG. 12a fixed with an elongated member,

FIGS. 13a to 13e show one possible way to form a loop from an elongated member,

FIGS. 14a to 14c show an elongated member with a retaining plate, and

FIG. 15 shows a cross-sectional view of elongated members which are used with cannulated screws and a retaining plate.

### DETAILED DESCRIPTION OF THE INVENTION

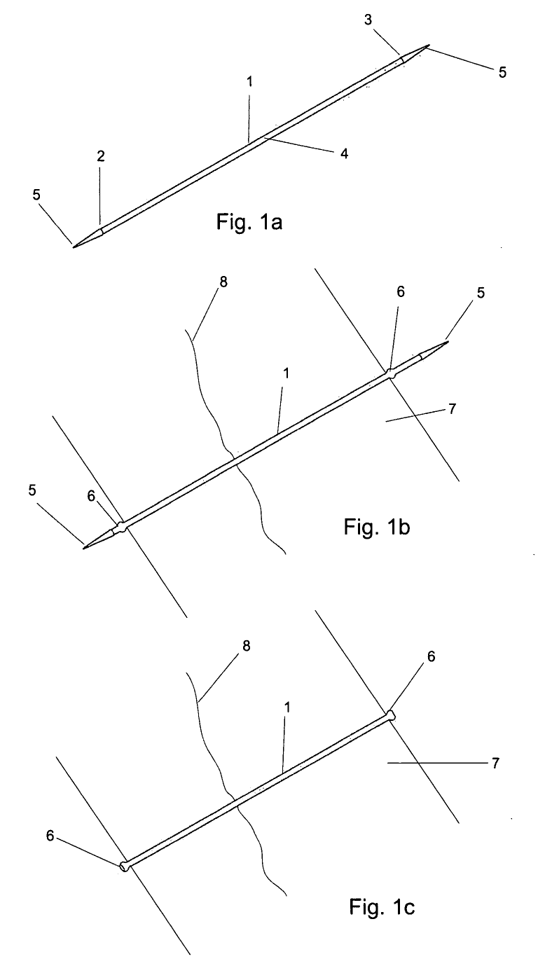

FIG. 1a shows a bioabsorbable elongated member which is a rod comprising a shaft 1. The cross-section of the rod may be cylindrical, square or any other suitable cross-section. The shaft 1 comprises a first end 2 and a second end 3 and an elongated part 4 therebetween. Needles 5 assisting in penetrating into a bone or tissue are attached to the both ends 2, 3 of the shaft 1. It is also possible that there is only one needle in one end of the shaft 1. The rod is delivered to its user as it is illustrated in FIG. 1a.

FIG. 1b shows the bioabsorbable elongated member, i.e. the rod of FIG. 1a, in such a situation in which the user has inserted the rod in a drillhole in a broken bone 7 (number 8 denotes the fracture) and he has formed two bulges 6 in the shaft 1. Providing both ends with needles 5 makes it easier to fix rather complicated fractures.

FIG. 1c shows the rod of FIGS. 1a and 1b when the excess of the shaft 1 has been cut off.

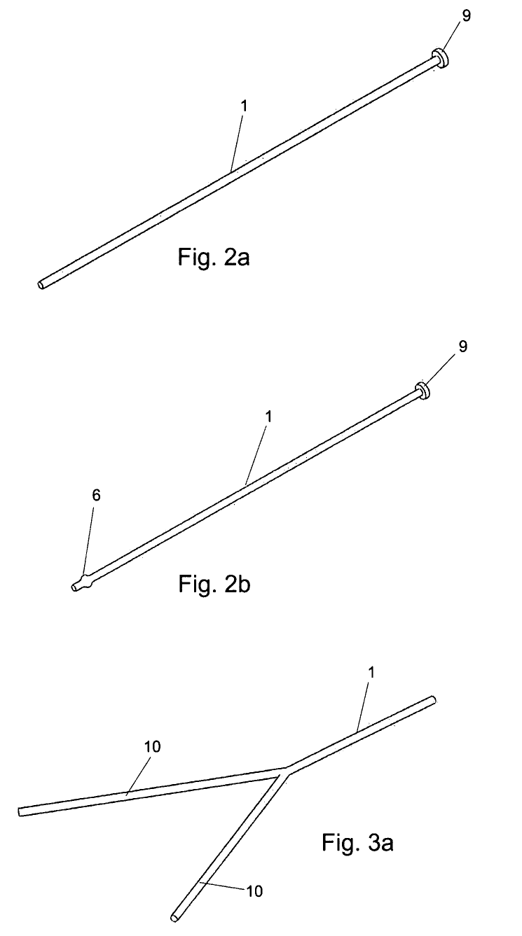

FIG. 2a shows a rod comprising a shaft 1 and a head 9. The head 9 is formed by pressing down. The rod is delivered to its user as it is illustrated in FIG. 2a.

FIG. 2b shows a situation in which the rod is inserted in a drillhole in a broken bone 7 (not shown). A bulge 6 forming a stopper is formed at the end of the shaft 1.

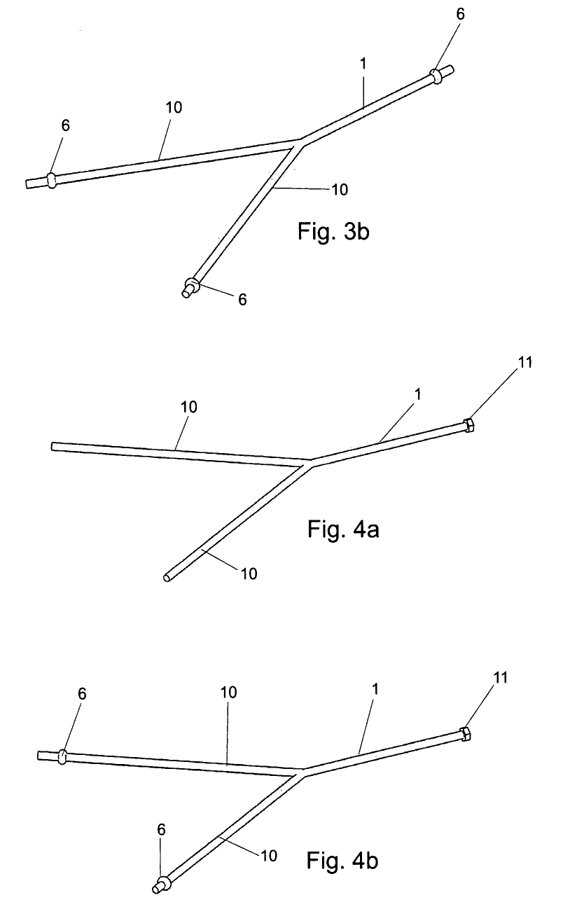

FIG. 3a shows an elongated member whose shaft 1 comprises branches 10. The branches 10 of the elongated member may be inserted in drillholes in a broken bone. After the branches have been positioned, bulges 6 are formed outside the bone 7 (not shown) as shown in FIG. 3b.

FIG. 4 shows another variation of the elongated member of FIG. 3. The elongated member comprises a pre-manufactured head 11 which may be manufactured by pressing down. Branches 10 are inserted in a drillhole in a bone and bulges 6 are formed in the branches.

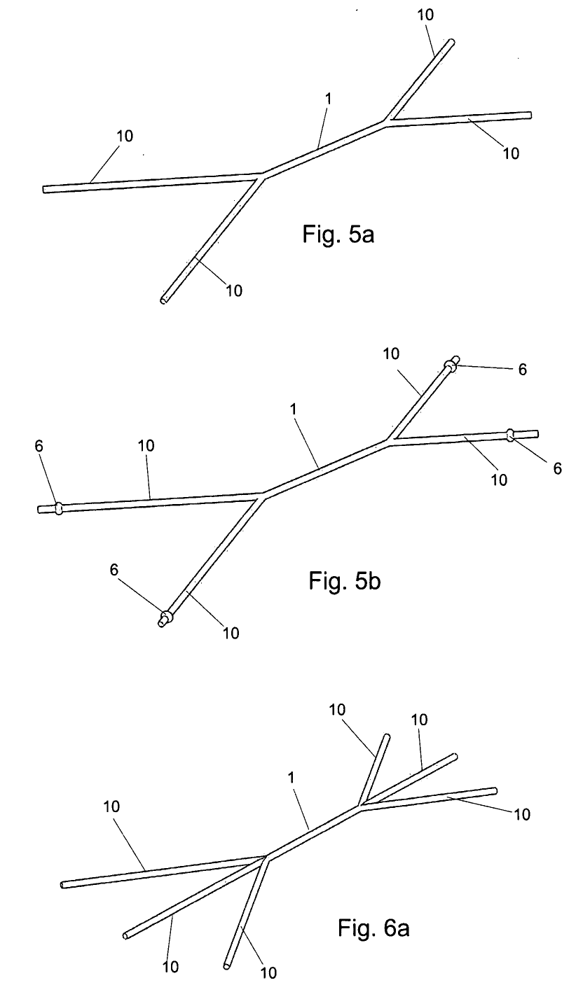

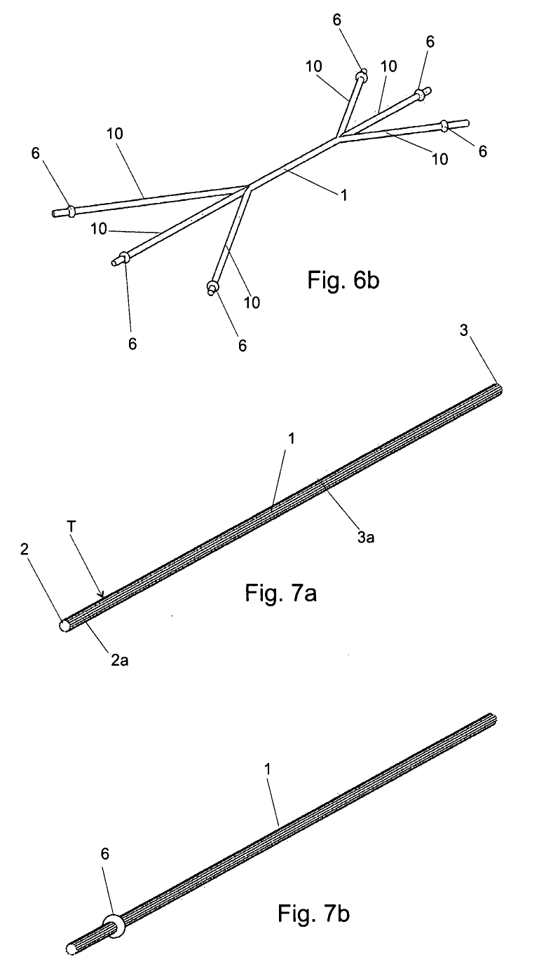

FIGS. 5 and 6 also show branched elongated members. FIGS. 5a and 6a show the elongated members before implanting, and FIGS. 5b and 6b show the elongated members after implanting. Bulges 6 are formed in each branch.

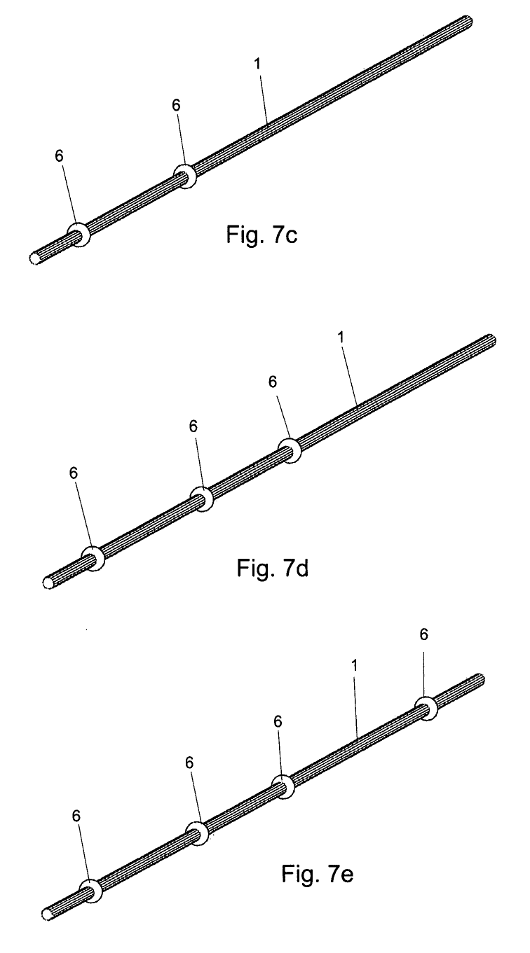

FIG. 7a shows an elongated member, such as a pin which has a grooved surface. FIG. 7a also shows a first end 2, a second end 3, a front end 2a, a back end 3a and a target point T. The front end 2a begins from the first end 2 and terminates at the target point T. The back end 3a begins from the target point T and terminates at the second end 3. A bulge 6 is formed at the target point T. FIGS. 7b to 7e show that in principle a bulge 6 can be formed in the pin wherever desired, i.e. there can be several target points T.

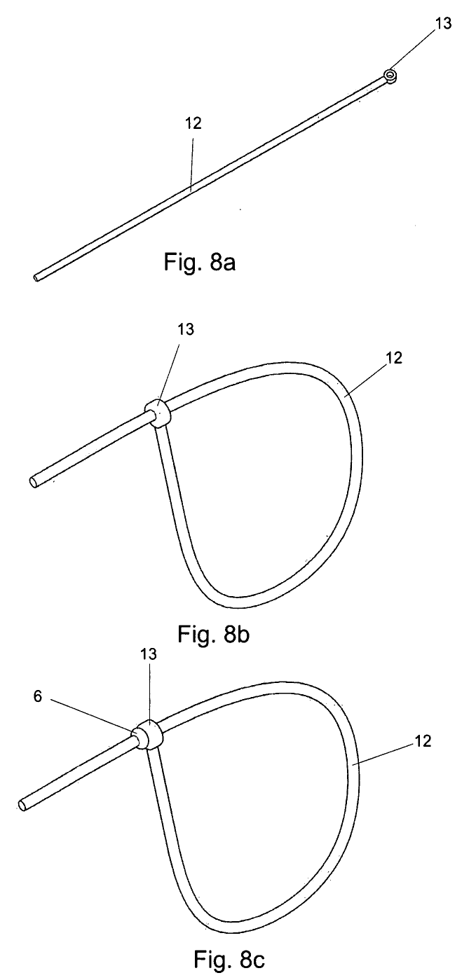

FIG. 8a shows an elongated member whose outer appearance resembles a cable tie. The elongated member has a flexible band section 12. The elongated member also comprises in its one end an eyelet 13 in which the other end can be threaded, as shown in FIG. 8b. After the elongated member has been inserted in a bone or tissue in a desired manner and threaded, a bulge 6 is formed outside the eyelet. Thus, the bulge forms a stopper as shown in FIG. 8c.

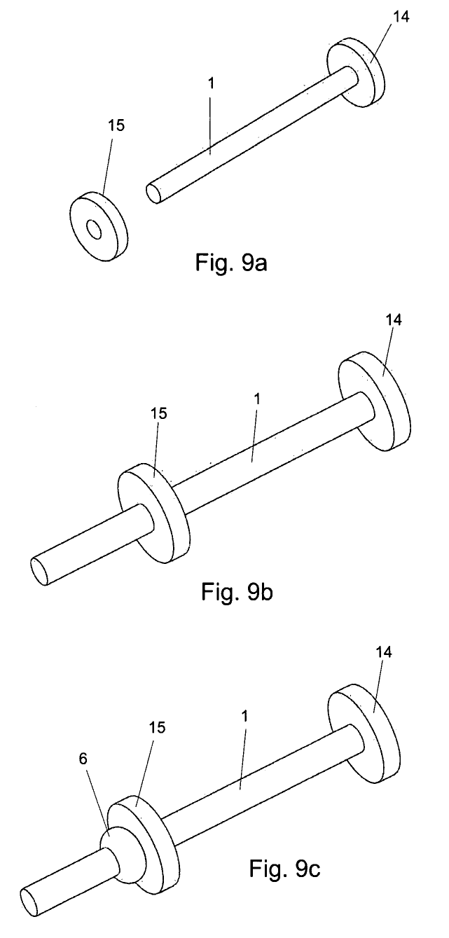

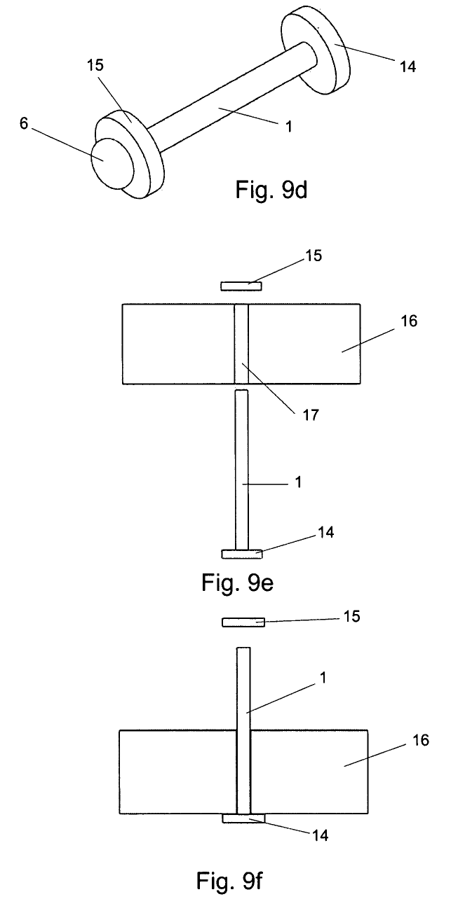

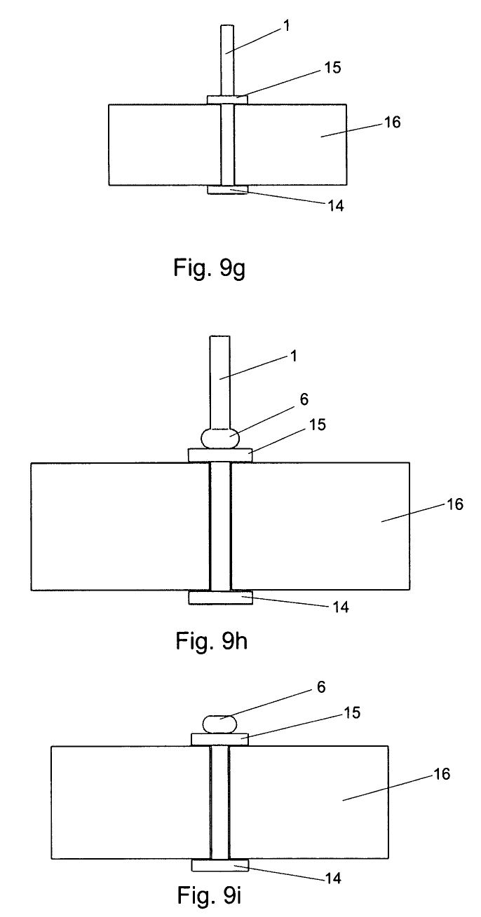

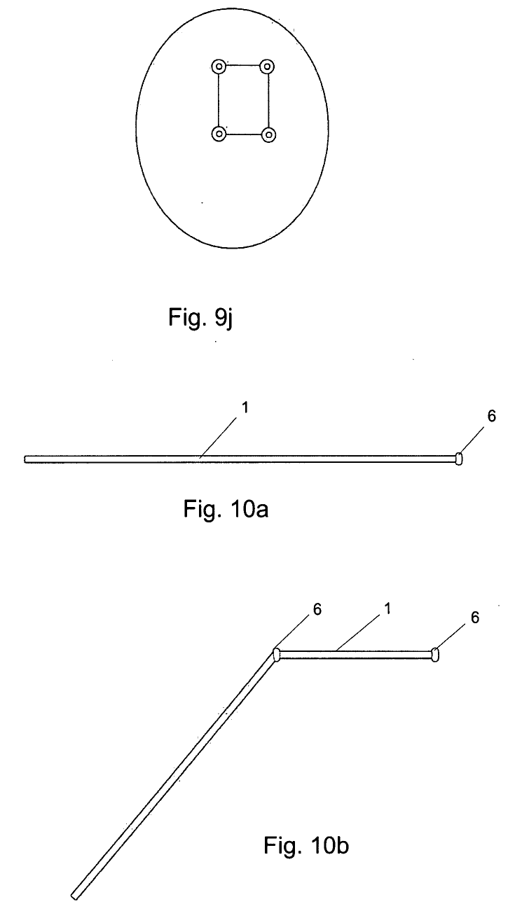

FIG. 9 shows an elongated member which is used e.g. in applications in which the cranium is fixed. The elongated member comprises a shaft 1 and a head 14. The shaft 1 of the elongated member is inserted through a drillhole in the cranium so that the head 14 of the elongated member is left under the cranium but over the dura. The head of the elongated member is a flat round plate as is natural due to the application. The diameter of the round plate may be at least 10 mm. The diameter of the shaft 1 may be around 10 mm.

After the elongated member has been inserted into the drillhole 17, the shaft 1 protrudes outside the cranium 16. An annular flat tightening ring 15 is inserted to the shaft 1, and a bulge 6 is formed outside the tightening ring 15, as shown in FIGS. 9c and 9h. The rest of the shaft 1 is cut as shown in FIGS. 9d and 9i.

FIG. 9j shows the cranium, which is fixed with the elongated members, from above. A piece of the cranial bone 16b has been separated from the rest of the cranium 16a in order to make surgical operations. There are four drillholes in each corner of the piece of the cranial bone in which elongated members are inserted. The elongated members are fastened at the edge of the piece of the cranial bone 16b. Bulges are formed on the elongated member so that they keep the piece of the cranial bone 16b in its position (see FIG. 9i).

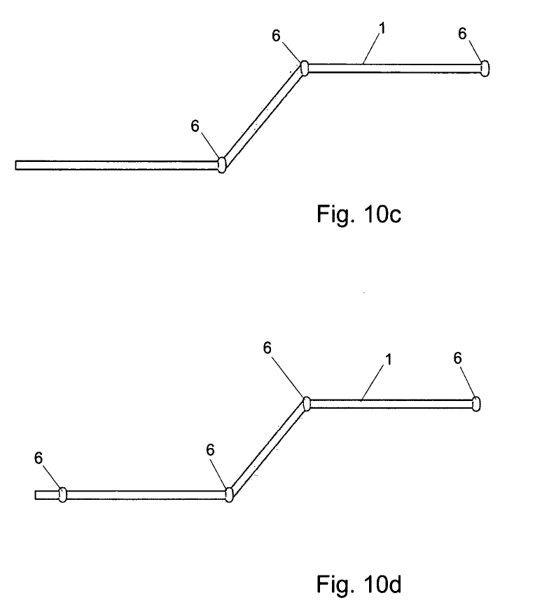

FIG. 10 shows an elongated member which is useful in applications in which the elongated member shall change its advancing direction. The elongated member may comprise a prefabricated head, or the head may be formed in situ. The advancing direction of the elongated member may be changed as desired as shown in FIGS. 10b and 10c by mechanically straining a certain point of the elongated member. When the material deforms at the point due to the heat caused by the mechanical straining, it is possible to form an angle in the elongated member. FIG. 10d shows a situation in which a desired length of the elongated member is used for the fixation and a bulge is formed at the end of the elongated member.

It is also possible to bend the elongated member without making the bulge 6 if the elongated member is sufficiently flexible. However, the bulge 6 increases the strength of the elongated member in its bending direction.

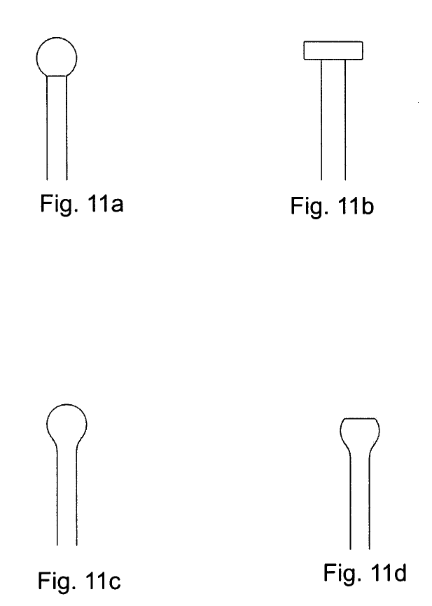

FIG. 11 shows variations of head designs of an elongated member. FIG. 11a shows a spherical head, FIG. 11b shows a flat round head, and FIG. 11c shows a spherical head whose joint with the shaft has been made gentle. FIG. 11d shows a truncated spherical head whose joint with the shaft has been made gentle.

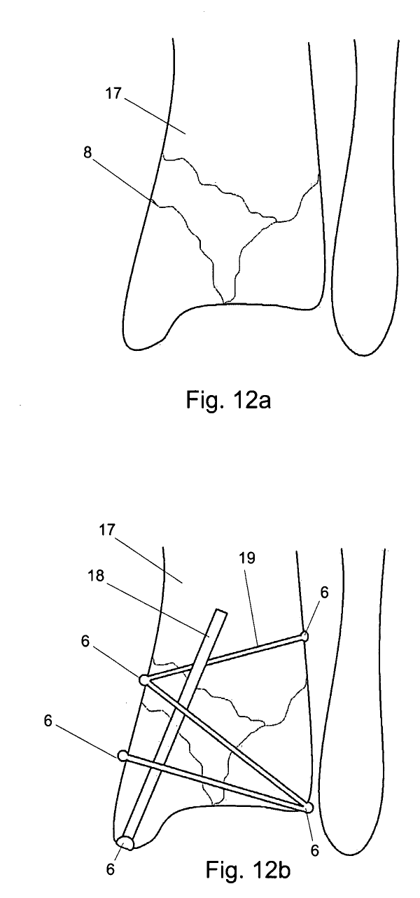

FIG. 12a shows fractures 8 in the distal end of tibia 17. FIG. 12b shows how the fractures 8 have been fixed with elongated members 18 and 19. The elongated member 18 is a pin which has been inserted in drillholes penetrating through the tibia 17. The elongated member 19 is a rod which has been inserted in a drillhole and whose advancing direction has been changed by forming bulges 6 outside the tibia 17.

The tibia is an example about possible targets of the elongated member. The principle of fixation is also the same with fractures in other bones: The elongated member is inserted in a drillhole or drillholes in a bone, and bulges are formed in the elongated member outside the bone in order to use them as stoppers or bending points.

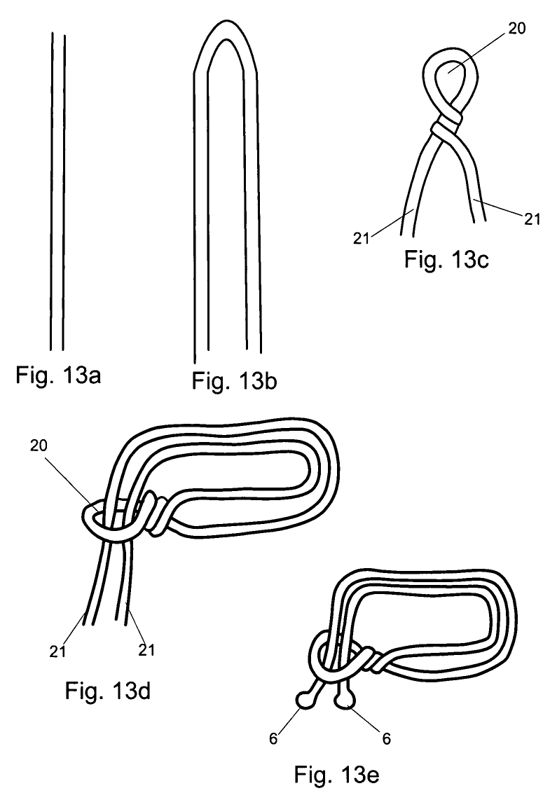

FIGS. 13a to 13e show one possible way to form a loop 20 from an elongated member. The straight elongated member of FIG. 13a is bent (FIG. 13b) and twisted (FIG. 13c) so that the loop 20 and two elongated tails 21 are formed. The legs 21 of the elongated member are threaded into the loop 20 (FIG. 13d). Bulges 6 are formed on the tails 21, and the tails 21 are cut underneath the bulges 6. The bulges 6 prevent the tails 21 from slipping out the loop 20.

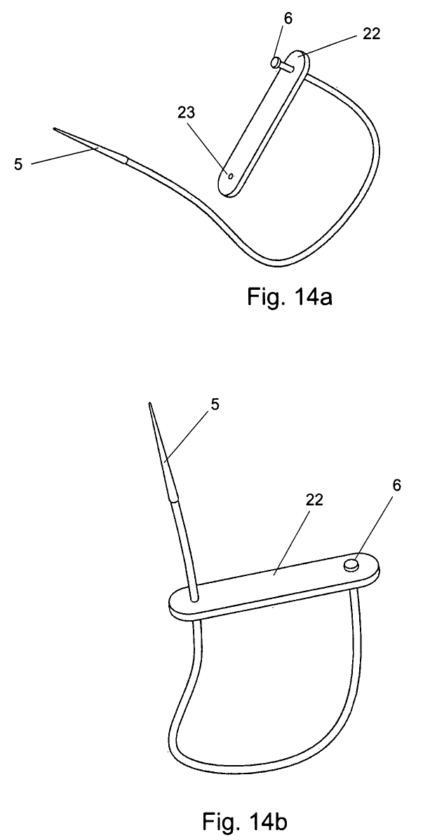

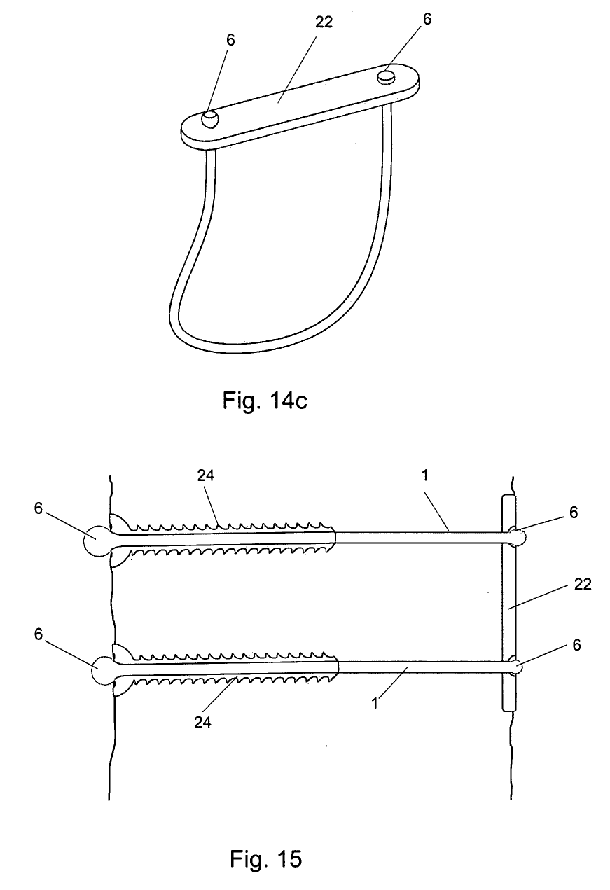

FIGS. 14a to 14c show an elongated member with a retaining plate 22. It is possible to form a bulge 6 on the elongated member before inserting the elongated member into a bone and/or tissue, or the bulge 6 can be formed after inserting the elongated member. The elongated member may be provided with a needle 5. After the elongated member has been inserted it forms either a loop around the bone and/or tissue, or it has been inserted in a drillhole or drillholes in the bone. It can be used for example for the fixation of the sternum. The ends of the elongated member are threaded through holes 23 in the retaining plate 22 and they are provided with the bulges 6. The elongated member is cut from the side of the bulge 6. The retaining plate 22 secures that the bulges 6 are prevented from slipping out from their position.

FIG. 15 shows elongated members which are used with cannulated screws 24 and a retaining plate 22. The cannulated screws 24 have been inserted in a bone and the elongated members are inserted via drillholes through the cannulated screws 24 and holes in the retaining plate 22. The ends of the elongated members are provided with bulges 6 which act as local stoppers. The cannulated screws 24 and the retaining plate 22 secures that the bulges 6 do not slip into the drillholes.

On the basis of the above description of the present invention and certain specific embodiments thereof, it will be readily apparent to those skilled in the art that many variations and modifications may be made to the present invention

1. A bioabsorbable elongated member, comprising:
a first end;
a second end; and
an elongated part therebetween, the elongated part comprising a front end, a back end and a target point in which the front end terminates and the back end begins, the bioabsorbable elongated member possessing a predetermined orientation, wherein the orientation of the bioabsorbable elongated member is alterable so that a bulge is formed in the bioabsorbable elongated member by straining the elongated member mechanically, thermally or thermomechanically at the target point, the bulge forming a local stopper of the bioabsorbable elongated member, or a part of a local stopper, or a bending point of the bioabsorbable elongated member.

2. The bioabsorbable elongated member according to claim 1, wherein the bioabsorbable elongated member is radially expandable so that a bulge is formed in the bioabsorbable elongated member by moving the front end and the back end repeatedly with regard to each other at the target point, the bulge forming a local stopper of the bioabsorbable elongated member, or a part of a local stopper, or a bending point of the bioabsorbable elongated member.

3. The bioabsorbable elongated member according to claim 1, wherein the bioabsorbable elongated member is radially expandable so that a bulge is formed in the bioabsorbable elongated member by heating the elongated member at the target point, the bulge forming a local stopper of the bioabsorbable elongated member, or a part of a local stopper, or a bending point of the bioabsorbable elongated member.

4. The bioabsorbable elongated member according to claim 1, wherein in that the bioabsorbable elongated member is radially expandable so that a bulge is formed in the bioabsorbable elongated member by mechanical straining and heating of the elongated member at the target point, the bulge forming a local stopper of the bioabsorbable elongated member, or a part of a local stopper, or a bending point of the bioabsorbable elongated member.

5. The bioabsorbable elongated member according to claim 1, further comprising:
a shaft.

6. The bioabsorbable elongated member according to claim 1, wherein the bioabsorbable elongated member comprises branches.

7. The bioabsorbable elongated member according to claim 1, wherein the bioabsorbable elongated member comprises a band.

8. The bioabsorbable elongated member according to claim 7, wherein the band comprises a loop at one end of the band.

9. The bioabsorbable elongated member according to claim 1, wherein the bioabsorbable elongated member comprises a separate tightening ring or a separate retaining plate.

10. The bioabsorbable elongated member according to claim 1, wherein the bioabsorbable elongated member comprises a needle or a bore bit.

11. The bioabsorbable elongated member according to claim 1, wherein the bioabsorbable elongated member comprises a copolymer of lactide and glycolide.

12. The bioabsorbable elongated member according to claim 11, wherein the bioabsorbable elongated member comprises 70 to 85 wt.-% of lactide and 15 to 30 wt.-% glycolide.

13. A method for forming a local stopper, a part of a local stopper, or a bending point in a bioabsorbable elongated member comprising a front end and a back end, the method comprising
determining a target point where the front end terminates and the back end begins, and
altering the orientation of the bioabsorbable elongated member so that a bulge is formed in the bioabsorbable elongated member by mechanical, thermal or thermomechanical straining of the elongated member at the target point, the bulge forming a local stopper of the bioabsorbable elongated member, or a part of a local stopper, or a bending point of the bioabsorbable elongated member.

14. The method according to claim 13, further comprising:
determining a target point where the front end terminates and the back end begins, and
moving the front end and the back end repeatedly with regard to each other at the target point, thereby forming a bulge used as the local stopper, the part of the local stopper, or the bending point.

15. The method according to claim 13, wherein before forming the bulge, a tightening ring or a retaining plate is inserted on the bioabsorbable elongated member.

16. The method according to claim 13, wherein after forming the bulge, the excess of the bioabsorbable elongated member is cut and/or pressed down.
